(19) United States
(12) Patent Application Publication
(10) Pub. No.: US 2024/0237947 A1
(43) Pub. Date: Jul. 18, 2024

(54) INDWELLING SENSING PROBE FOR VASCULAR SENSING AND RELATED SYSTEMS AND METHODS

(71) Applicant: Becton, Dickinson and Company, Franklin Lakes, NJ (US)

(72) Inventor: Jonathan Karl Burkholz, Salt Lake City, UT (US)

(21) Appl. No.: 18/503,012

(22) Filed: Nov. 6, 2023

Related U.S. Application Data
(60) Provisional application No. 63/438,878, filed on Jan. 13, 2023.

(51) Int. Cl. A61B 5/00; A61B 5/0205

(52) U.S. Cl. CPC A61B 5/6848; A61B 5/0205; A61B 5/14503

(57) ABSTRACT

A vascular access system may include an introducer needle, which may include a sharp distal tip, a proximal end, and an introducer needle lumen. The vascular access system may include a sensing probe slidably disposed within the introducer needle lumen. A distal end of the sensing probe may include one or more sensors and may be disposed within the introducer needle lumen in an initial configuration. The introducer needle may be configured to retract in response to the distal end of the sensing probe being advanced beyond the sharp distal tip. A method may include inserting the introducer needle into a vein or an artery of a patient, advancing the distal end of the sensing probe beyond the sharp distal tip such that the introducer needle is retracted, and the sensing probe remains in the vein or the artery, and receiving sensor data from the one or more sensors.

### RELATED APPLICATIONS

This application claims the benefit of U.S. Provisional Patent Application No. 63/438,878, which was filed on Jan. 13, 2023, which is incorporated herein in its entirety.

### BACKGROUND

Current arterial catheter systems provide monitoring of a patient's arterial hemodynamic characteristics. They also provide vascular access to collect arterial blood for arterial blood gas (ABG) testing and analysis. However, the current arterial catheter systems and related methods have drawbacks, including significant blood exposure risk and complicated and time-consuming collection and preservation of ABG samples. There is also a risk of health complications such as catheter-related bloodstream infections (CRBSI) and a need for proper line and device flushing.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to an indwelling sensing probe for vascular sensing, as well as related systems and methods. In some embodiments, the indwelling sensing probe may address one or more of the drawbacks of the current arterial catheter systems. In some embodiments, the indwelling sensing probe may provide continuous or intermittent hemodynamic and blood characteristic measurement and analysis.

In some embodiments, a vascular access system may include an introducer needle, which may include a sharp distal tip, a proximal end opposite the sharp distal tip, and an introducer needle lumen extending through the sharp distal tip and the proximal end of the introducer needle. In some embodiments, the vascular access system may include a sensing probe slidably disposed within the introducer needle lumen. In some embodiments, the sensing probe may be configured to be indwelling or left within vasculature of a patient for continuous vascular sensing after the introducer needle is withdrawn from the vasculature.

In some embodiments, a distal end of the sensing probe may be disposed within the introducer needle lumen in an initial configuration of the vascular access system. In some embodiments, the introducer needle may be configured to retract in response to the distal end of the sensing probe being advanced beyond the sharp distal tip. In some embodiments, the distal end of the sensing probe may include one or more sensors.

In some embodiments, the vascular access system may include a housing, which may include a longitudinal passage. In some embodiments, the introducer needle may be slidably disposed within the longitudinal passage of the housing. In some embodiments, the introducer needle may extend from a distal end of the housing in the initial configuration of the vascular access system.

In some embodiments, the vascular access system may include a sensing probe hub. In some embodiments, the sensing probe may extend distally from the sensing probe hub. In some embodiments, the introducer needle may be fully retractable within the sensing probe hub and the housing when the sensing probe hub is fully advanced with respect to the housing. In some embodiments, a proximal end of the sensing probe hub may be coupled to or configured to couple to a cable configured to transmit sensor data from the sensor. In some embodiments, the vascular access system may include a wireless adapter configured to transmit sensor data from the sensor. In some embodiments, the vascular access system may include a stabilization platform coupled to the sensing probe hub. In some embodiments, the wireless adapter may be coupled to the stabilization platform.

In some embodiments, the sensing probe may include a guidewire. In some embodiments, the sensor may be configured to measure one or more physiological or hemodynamic parameters. For example, the sensor may be configured to measure one or more of blood pressure, temperature, pH, oxygen, and oxygen saturation. Additionally or alternatively, the sensor may be configured to measure one or more other physiological parameters. In some embodiments, the vascular access system may include a compressible sleeve surrounding the sensing probe and coupled to the sensing probe hub.

In some embodiments, the vascular access system may include a protective tube extending from the sensing probe hub. In some embodiments, the sensing probe may be disposed within the protective tube and spaced apart from the protective tube such that fluid, such as blood, for example, may flow between the protective tube and the sensing probe. In some embodiments, the sensing probe hub may include a port. In some embodiments, the vascular access system may include an extension tube integrated with the port. In some embodiments, fluid may be configured to flow proximally through the protective tube between the protective tube and the sensing probe and into the extension tube. In some embodiments, the introducer needle may be held in place in the initial configuration by a releasable latch. In some embodiments, the sensing probe may be configured to release the releasable latch to allow the introducer needle to retract.

In some embodiments, a method of venous or arterial sensing may include inserting the introducer needle of the vascular access system into a vein or an artery of a patient. In some embodiments, the method may include advancing the distal end of the sensing probe beyond the sharp distal tip such that the introducer needle is retracted, and the sensing probe remains in the vein or the artery. In some embodiments, the method may include receiving sensor data from the sensor. In some embodiments, the sensor data may include at least one blood characteristic, such as one or more physiological or hemodynamic parameters. In some embodiments, the at least one blood characteristic may be selected from: blood pressure, temperature, pH, oxygen, blood gas, and oxygen saturation. In some embodiments, the at least one blood characteristic may include one or more other physiological or hemodynamic parameters.

In some embodiments, the method may include processing the sensor data from the sensor to determine a parameter. In some embodiments, the parameter may include an indication of an onset of a health complication or an indication of an onset of a health improvement. In some embodiments, the parameter may be determined using artificial intelligence.

In some embodiments, the vascular access system may include a connector disposed at a distal end of the housing and configured to couple to a catheter assembly. In some embodiments, the sensing probe hub may be configured to advance distal to the distal end of the housing in response to movement of the sensing probe hub towards the housing. In some embodiments, the connector may include a blunt plastic cannula or a male luer. In these embodiments, the vascular access system may not include the introducer needle.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example vascular access system, according to some embodiments;

FIG. 1B is an upper perspective view of the vascular access system, illustrating an initial configuration, according to some embodiments;

FIG. 1C is an upper perspective view of the vascular access system, illustrating an advanced configuration with an example sensing probe fully advanced and an example introducer needle fully retracted, according to some embodiments;

FIG. 1D is an upper perspective view of the vascular access system, illustrating the initial configuration, according to some embodiments;

FIG. 1E is a cross-sectional view of the vascular access system, illustrating the initial configuration, according to some embodiments;

FIG. 1F is an exploded view of the vascular access system, according to some embodiments;

FIG. 1G is an upper perspective view of the introducer needle, according to some embodiments;

FIG. 1H is a top view of a proximal end of the introducer needle, according to some embodiments;

FIG. 1I is a side view of a proximal end of the introducer needle, according to some embodiments;

FIG. 1J is an upper perspective view of the introducer needle and an example coil, according to some embodiments;

FIG. 1K is a cross-sectional view of the vascular access system, illustrating the initial configuration, according to some embodiments;

FIG. 1L is a cross-sectional view of the vascular access system, illustrating an example sensing probe hub advanced from the initial configuration to release the introducer needle, according to some embodiments;

FIG. 1M is a cross-sectional view of the vascular access system, illustrating the advanced configuration, according to some embodiments;

FIG. 2 is an upper perspective view of the vascular access system, illustrating an example compressible sleeve, according to some embodiments;

FIG. 3A is an upper perspective view of the vascular access system, illustrating an example protective sleeve and fluid pathway, according to some embodiments;

FIG. 3B is a cross-sectional view of the vascular access system, illustrating the protective sleeve and fluid pathway, according to some embodiments;

FIG. 3C is a cross-sectional view of the vascular access system, illustrating the initial configuration, according to some embodiments;

FIG. 3D is a cross-sectional view of the vascular access system, illustrating the sensing probe hub advanced from the initial configuration to release the introducer needle, according to some embodiments;

FIG. 3E is a cross-sectional view of the vascular access system, illustrating the advanced configuration, according to some embodiments;

FIG. 4 is an upper perspective view of an example securement dressing placed over top of a portion of the vascular access system, according to some embodiments; and

FIG. 5 is an upper perspective view of the vascular access system, illustrating an example connector at a distal end of an example housing, according to some embodiments.

### DESCRIPTION OF EMBODIMENTS

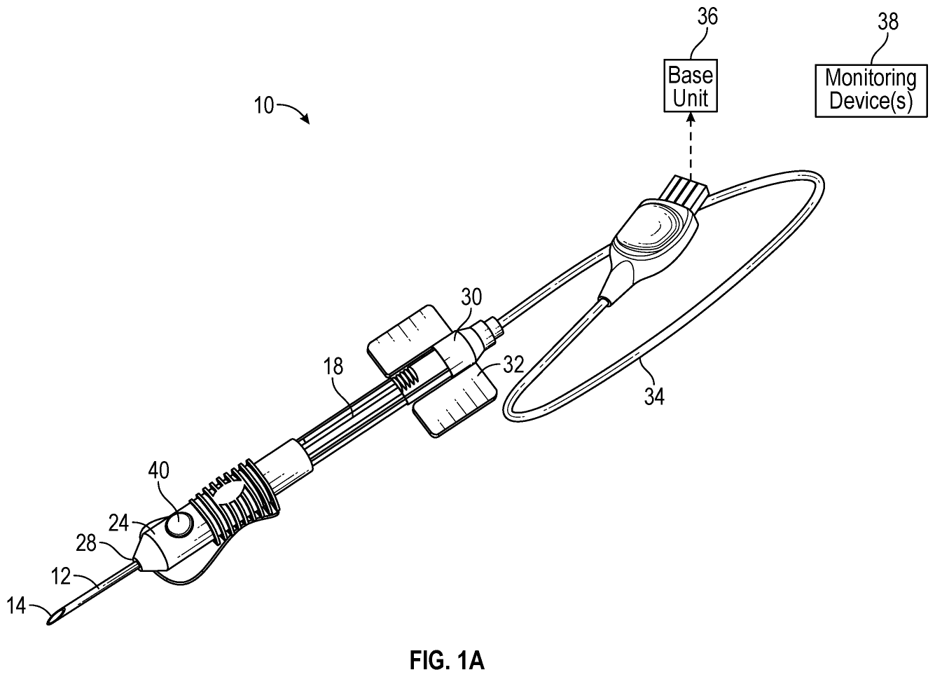

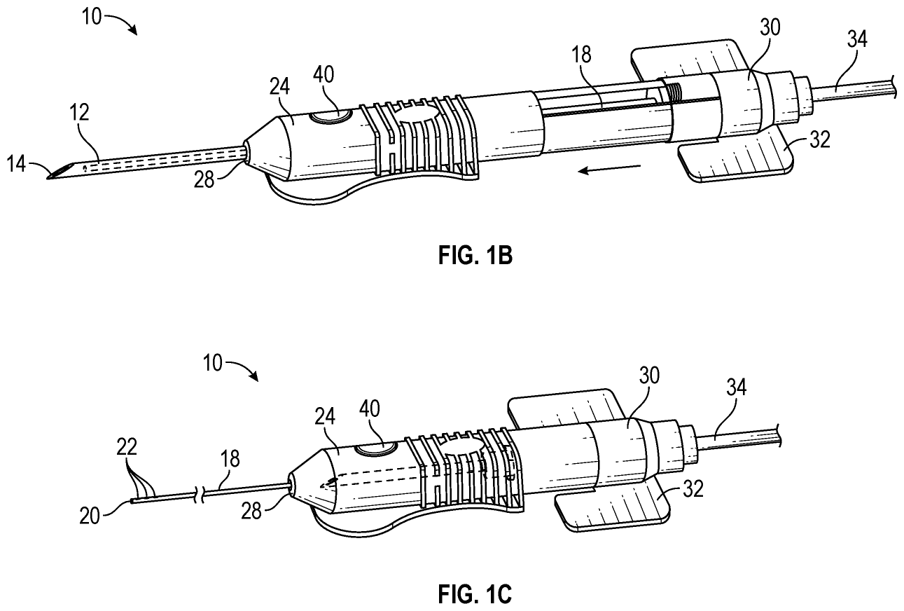

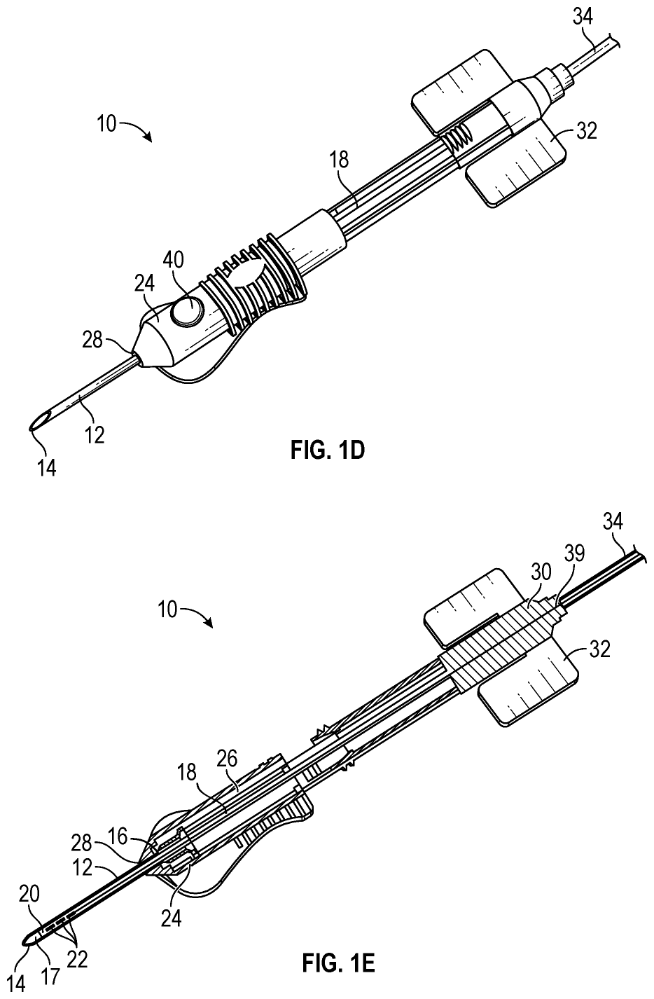

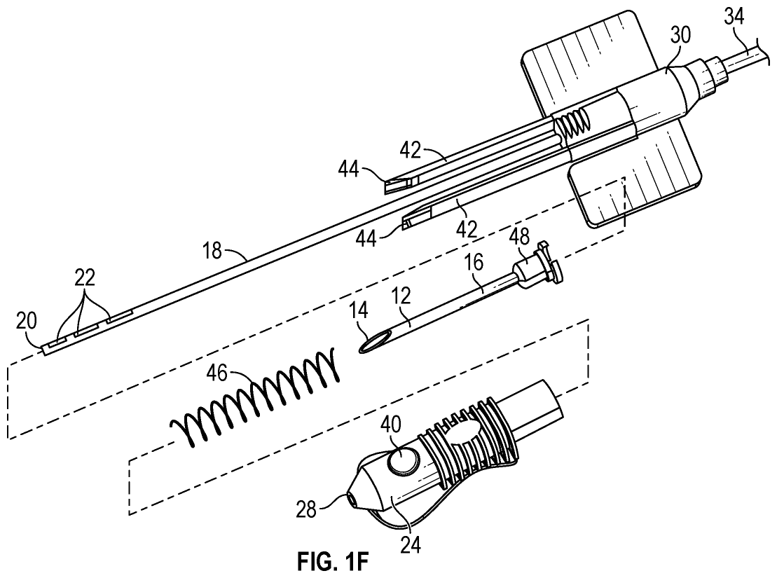

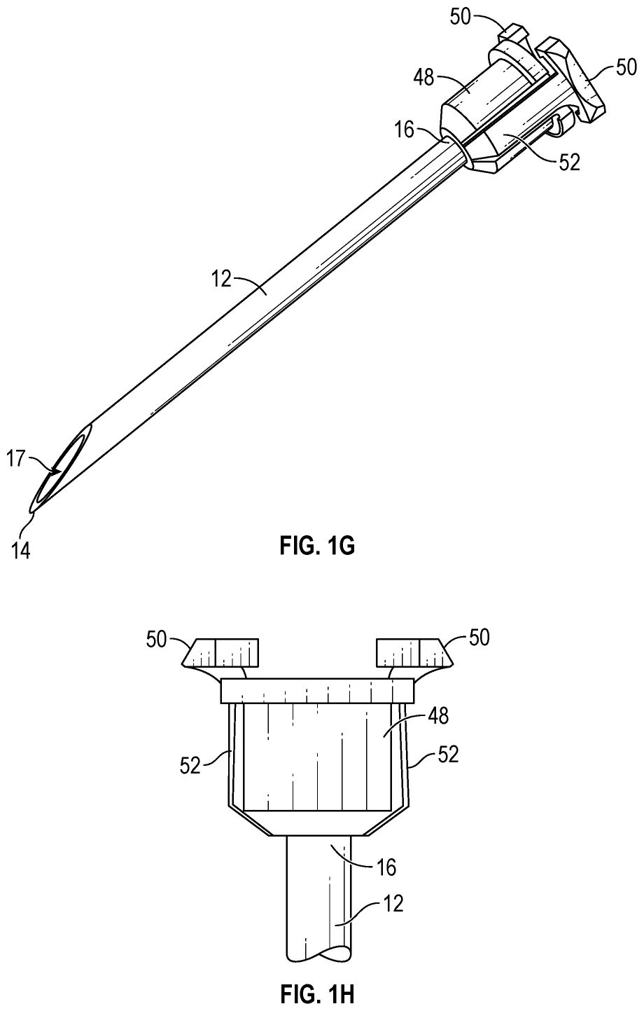

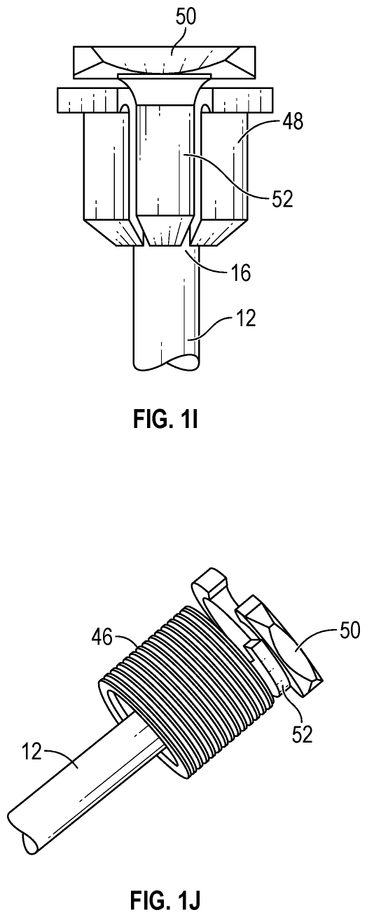

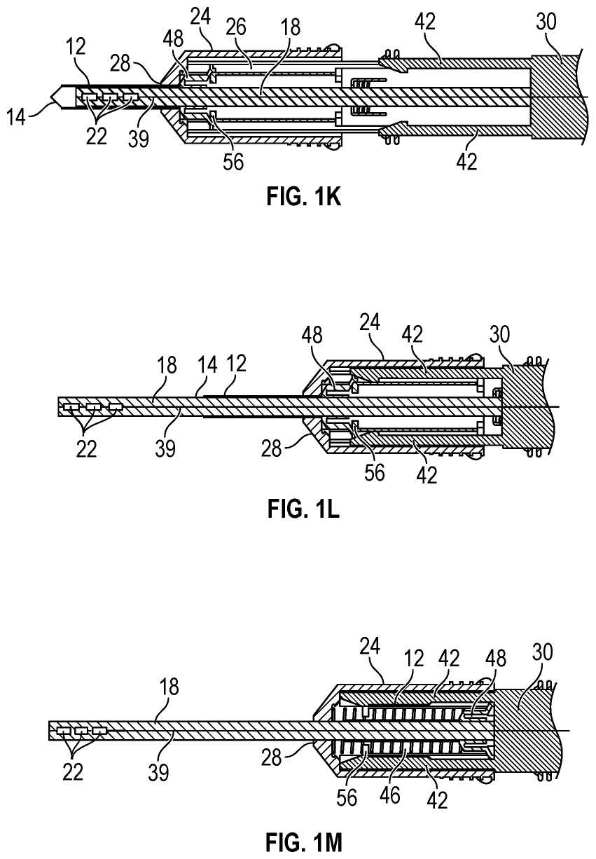

Referring now to FIGS. 1A-1E, in some embodiments, a vascular access system 10 may include an introducer needle 12, which may include a sharp distal tip 14, a proximal end 16 opposite the sharp distal tip 14, and an introducer needle lumen 17 extending through the sharp distal tip 14 and the proximal end 16 of the introducer needle 12. In some embodiments, the vascular access system 10 may include a sensing probe 18 slidably disposed within the introducer needle lumen 17. In some embodiments, the sensing probe 18 may be configured to be indwelling or left within vasculature of a patient for vascular sensing after the introducer needle 12 is withdrawn from the vasculature. For example, the sensing probe 18 may be indwelling within a vein or an artery and configured to sense one or more blood characteristics therein.

In some embodiments, a distal end 20 of the sensing probe 18 may be disposed within the introducer needle lumen 17 in an initial configuration of the vascular access system 10, illustrated, for example, in FIGS. 1A-1B and 1D-1E. In some embodiments, the introducer needle 12 may be configured to retract in response to the distal end 20 of the sensing probe 18 being advanced beyond the sharp distal tip 14. In some embodiments, the distal end 20 of the sensing probe 18 may include one or more sensors 22, which may be integrated within the sensing probe 18. In some embodiments, the sensors 22 may be arranged in various patterns and configurations.

In some embodiments, the sensors 22 may be configured to measure one or more physiological or hemodynamic parameters. For example, the sensors 22 may be configured to measure one or more of blood pressure, temperature, pH, oxygen, and oxygen saturation (SpO2). Additionally or alternatively, in some embodiments, the sensors 22 may be configured to measure one or more other physiological or hemodynamic parameters. In some embodiments, the sensors 22 may be configured to measure one or more other blood characteristics. In some embodiments, after retraction of the introducer needle 12, the sensing probe 18 may remain within the vein or artery of the patient to provide short-term or long-term monitoring of the one or more blood characteristics.

In some embodiments, the vascular access system 10 may include a housing 24, which may include a longitudinal passage 26. In some embodiments, the introducer needle 12 may be slidably disposed within the longitudinal passage 26 of the housing 24. In some embodiments, the introducer needle 12 may extend from a distal end 28 of the housing 24 in the initial configuration of the vascular access system 10. In some embodiments, the vascular access system 10 may include a sensing probe hub 30. In some embodiments, the sensing probe 18 may extend distally from the sensing probe hub 30.

In some embodiments, the vascular access system 10 may include a stabilization platform 32 coupled to the sensing probe hub 30. In some embodiments, the stabilization platform 32 may facilitate securement of the sensing probe hub 30 to a patient after the sensing probe 18 is advanced distally. In some embodiments, the stabilization platform 32 may include a wing or two opposing wings. In some embodiments, two opposing wings may each extend from the sensing probe hub 30.

In some embodiments, in a wired configuration, a proximal end of the sensing probe hub 30 may be coupled to or configured to couple to a cable 34 configured to transmit sensor data from the sensors 22. In further detail, in some embodiments, the cable 34 may be removably coupled to the sensing probe hub 30. In other embodiments, the cable 34 may be integrated with the sensing probe hub 30. In some embodiments, the vascular access system may include a wireless adapter configured to transmit sensor data from the sensors 22, as will be explained later in further detail.

In some embodiments, the vascular access system 10 may include a base unit 36 to which the cable 34 may be connected to enable the base unit 36 and the sensors 22 to communicate. In some embodiments, the base unit 36 may include any device that includes circuitry for communicating with the sensors 22. In some embodiments, the base unit 36 may provide power to the sensors 22. In some embodiments, the base unit 36 may directly process sensor data received from the sensors 22, while in other embodiments, the base unit 36 may receive the sensor data and forward the sensor data to another device for processing. In some embodiments, the base unit 36 may be connected to one or more other devices to allow users of the one or more other device to control and/or receive sensor data from the sensors 22.

In some embodiments, the vascular access system 10 may include a monitoring device 38. In some embodiments, the monitoring device 38 may include any device having a display on which the sensor data may be displayed and/or on which information obtained from the sensor data may be displayed. As examples, the monitoring device 38 may include a smart phone, a tablet, a laptop, a desktop, a thin client, a television, a dedicated display device, an infusion pump, a patient vital sign monitor, an arterial monitor, etc. In some embodiments, a monitoring device 38 may function as the base unit 36. In some embodiments, the monitoring device 38 and/or the base unit 36 could also be configured to interface with one or more separate computing systems such as a system for storing patient data.

In some embodiments, to perform venous or arterial sensing, the introducer needle 12 of the vascular access system 10 may be inserted into a vein or an artery of a patient. In some embodiments, the distal end 20 of the sensing probe 18 may then be advanced beyond the sharp distal tip 14 such that the introducer needle 12 is retracted, and the sensing probe 18 remains in the vein or the artery. In some embodiments, sensor data may be received from the sensors 22 (such as by the base unit 36, for example). In some embodiments, the sensor data may include at least one blood characteristic.

In some embodiments, the sensor data from the sensors 22 may be processed to determine a parameter. In some embodiments, the parameter may be determined using artificial intelligence. In some embodiments, the parameter may include an indication of an onset of a health complication, an indication of an onset of a health improvement, a change in a measured parameter, or another health parameter. In some embodiments, the sensors 22 may collect the sensor data continuously, intermittently, or selectively.

In some embodiments, the sensing probe 18 may be rigid or semi-rigid. In some embodiments, the sensing probe 18 may include a rod, a guidewire, or another suitable probe. In some embodiments, sensing probe 18 may be constructed of nitinol or another type of metal wire. In some embodiments, one or more sensor wire leads 39 and/or one or more optical fibers may extend through the sensing probe 18 and/or the cable 34 to allow the sensors 22 to communicate with the base unit 36. In some embodiments, the sensing probe 18 may be constructed of plastic or another suitable material that may reduce a risk of damaging the vasculature. In these and other embodiments, the sensing probe 18 may include a one or more core wires for structure and durability during distal advancement. For example, the sensing probe 18 may include one or more nitinol core wires running along a length or an entire length of the sensing probe 18. In some embodiments, the one or more core wires may be disposed at a center axis of the sensing probe 18 or offset from the center axis. In some embodiments, the distal end 20 of the sensing probe 18 may be blunt and atraumatic to reduce a risk of the sensing probe 18 inducing damage or complications within the vasculature.

In some embodiments, the sensing probe 18 may be coated with one or more coatings to improve the performance and reduce a risk of complications such as development of a thrombus or probe-related blood stream infection. In some embodiments, a particular coating on the sensing probe 18 may include a silicon lube with or without an antimicrobial additive, such as, for example, chlorhexidine. In some embodiments, the sensing probe 18 may be coated with an anti-thrombogenic coating and/or an anti-microbial coating. In some embodiments, the anti-thrombogenic coating and/or the anti-microbial coating may include one or more polymer additives.

In some embodiments, the vascular access system 10 may include any suitable needle retraction mechanism to facilitate retraction of the introducer needle 12 while leaving the sensing probe 18 within the vasculature. In some embodiments, the introducer needle 12 may be retracted manually or via another suitable method. One example needle retraction mechanism is described in U.S. Pat. No. 11,197,980, filed Apr. 29, 2021, entitled “RETRACTABLE NEEDLE CATHETER DELIVERY APPARATUS,” which is hereby incorporated by reference in its entirety.

In some embodiments, in the initial configuration of the vascular access system 10, the introducer needle 12 may be fully extended in a distal direction from the housing 24, and the sensing probe 18 may be fully retracted in a proximal direction within the housing 24. In this configuration, the introducer needle 12 is ready to be percutaneously inserted into the vasculature. In some embodiments, the sharp distal tip 14 may be inserted into a lumen of a target vein or artery, and entry may be confirmed by observing flashback in a window 40 formed in a wall of the housing 24.

Referring now to FIG. 1F, in some embodiments, a pair of arms 42 is attached to the sensing probe hub 30 and extend from the sensing probe hub 30 in a distal direction parallel to a longitudinal axis of the sensing probe 18. In some embodiments, a distal end of the arms 42 may terminate in a wedge tip 44 which is configured to release the introducer needle 12 from temporary attachment to the housing 24 as the sensing probe 18 is distally advanced within the longitudinal passage 26 of the housing 24. In some embodiments, the vascular access system 10 may include a coil 46 or other compression spring. As further seen in FIG. 1F, in some embodiments, the introducer needle 12 may include a shaft portion and a needle hub 48 at the proximal end 16 opposite the sharp distal tip 14.

Referring now to FIGS. 1G-1J, in some embodiments, the needle hub 48 may include a pair of engagement hooks or tangs 50 which serve to “releasably” lock the introducer needle 12 in the initial or distally forward configuration prior to retraction of the introducer needle 12 after the sensing probe 18 has been advanced. In some embodiments, the hooks or tangs 50 act as a releasable latch to hold the introducer needle 12 in place in the initial configuration, the sensing probe 18 being configured to release the releasable latch to allow the introducer needle 12 to retract.

In some embodiments, the needle hub 48 may include a cylindrical wall having a pair of opposed cantilever spring elements 52 formed therein. In some embodiments, the cantilever spring elements 52 may be attached to the cylindrical wall at their distal ends so that the engagement hooks or tangs 50 at their proximal ends maybe resiliently compressed inwardly to release the needle hub 48 from engagement with a wall of the housing 24. As illustrated, for example, in FIG. 1J, the coil 46 may be maintained in a compressed configuration prior to release of the introducer needle 12 for retraction into the housing 24. In some embodiments, release of the needle hub 48 from engagement with the housing 24 allows the coil 46 to decompress and drive the introducer needle 12 proximally so that it fully retracts into the housing 24.

Referring now to FIGS. 1K-1M, step-wise advancement of the sensing probe 18 and retraction of the introducer needle 12 into the housing 24 will be described, according to some embodiments. As illustrated, for example, in FIG. 1K, the sharp distal tip 14 of the introducer needle 12 extends fully distally of the housing 24 and is ready for insertion through the patient's skin into the vasculature. At this point, in some embodiments, the sensing probe 18 may be fully retracted in a proximal direction and held in place by engagement of the wedge tips 44 of the arms 42 with the housing 24.

In some embodiments, after the introducer needle 12 has been advanced into the vasculature, the sensing probe 18 may be advanced into the vasculature by distally pushing the sensing probe hub 30 into the housing 24. In some embodiments, as the wedge tips 44 are distally advanced, they may engage the engagement hooks or tangs 50 on the needle hub 48. In particular, inclined surfaces of the wedge tips 44 may engage beveled surfaces of the cantilever spring element 52 forcing the cantilever spring elements 52 inwardly and disengaging the engagement hooks or tangs 50 from retention slots 56 formed on the inner wall of the axial passage 203 of the housing 24. In some embodiments, once the tangs 50 are released from the retention slots 56, the needle hub 48 may be released to translate freely, and the coil 46 may decompress to translate the needle hub 48 proximally, retracting the introducer needle 12 fully into the housing 24. In some embodiments, the introducer needle 12 may be fully retractable within the sensing probe hub 30 and the housing 24 when the sensing probe hub 30 is fully advanced with respect to the housing 24, as illustrated, for example, in FIG. 1M.

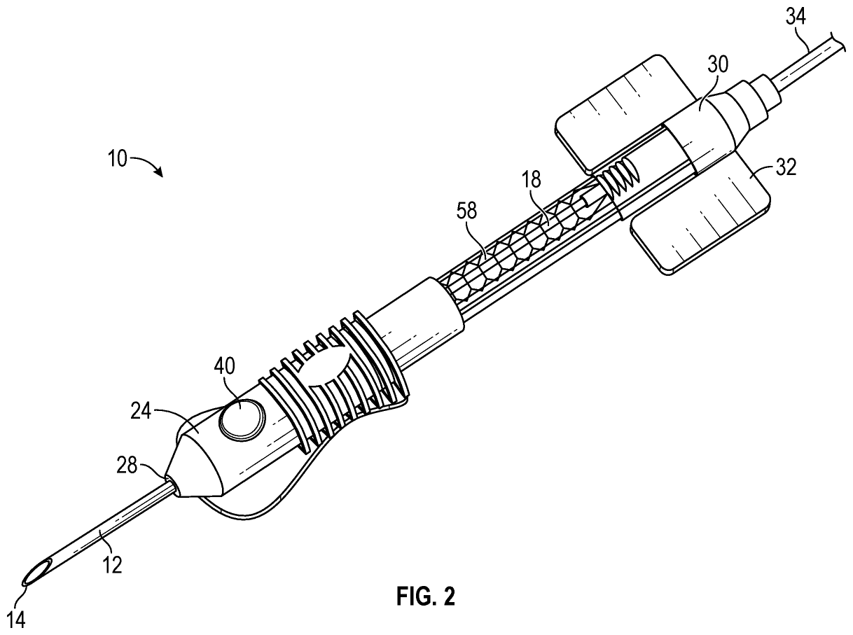

Referring now to FIG. 2, in some embodiments, the vascular access system 10 may include a compressible sleeve 58 surrounding the sensing probe 18. In some embodiments, a proximal end of the compressible sleeve 58 may be coupled to the sensing probe hub 30, and thus, the compressible sleeve 58 may compress in response to distal movement of the sensing probe hub 30 and advancement of the sensing probe 18. In some embodiments, a distal end of the compressible sleeve 58 may be coupled to the housing 24, a component within the housing 24, or another suitable location. In some embodiments, the compressible sleeve 58 may protect the sensing probe 18 from bacterial contamination and reduce a risk of introducing microbes into the vein or the artery during a process of placing the sensing probe 18 therein. In some embodiments, the compressible sleeve 58 may be corrugated or accordion-shaped, which may facilitate organized folding of the compressible sleeve 58 as it is compressed.

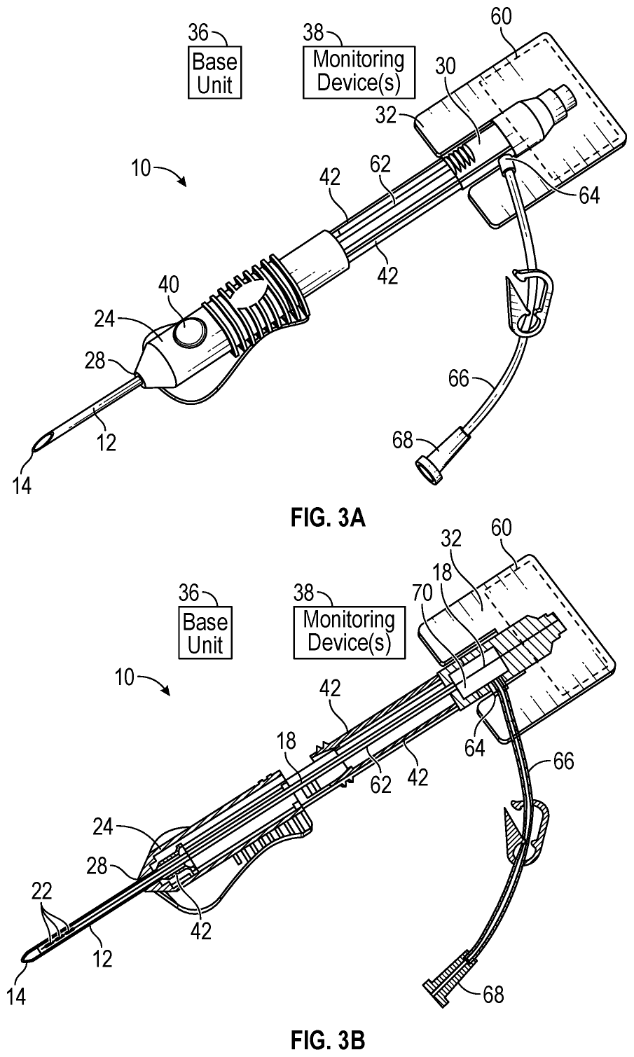

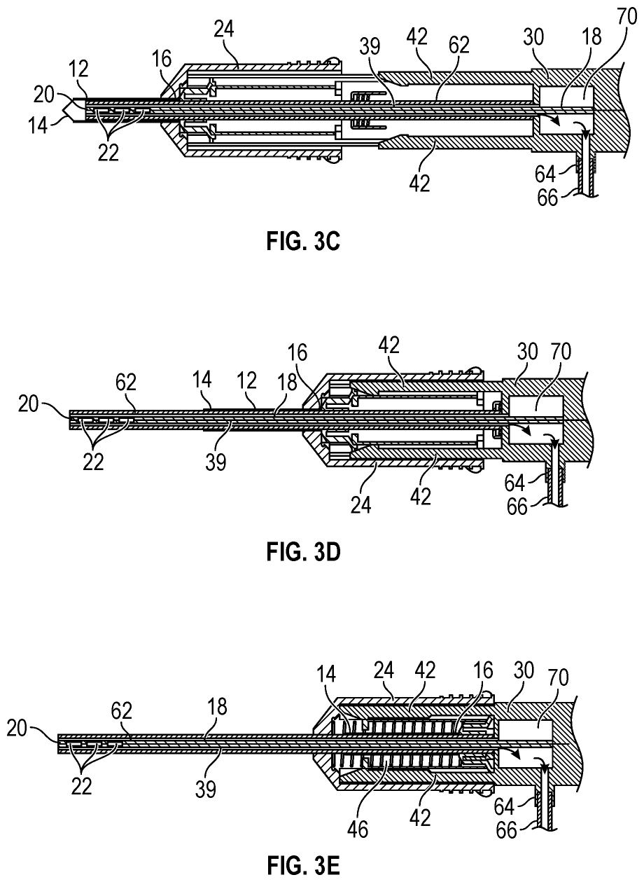

Referring now to FIGS. 3A-3E, in some embodiments, the vascular access system 10 may include a wireless adapter 60 configured to transmit sensor data from the sensors 22. In some embodiments, the wireless adapter 60 may be coupled to and/or integrated with the stabilization platform 32. In some embodiments, the wireless adapter 60 may be disposed within a housing that may be coupled to the sensing probe hub 30.

In some embodiments, the wireless adapter 60 may wirelessly connect the sensors 22 to the base unit 36. In some embodiments, the wireless adapter 60 may be used in place of the cable 34, illustrated, for example, in FIGS. 1A-1F. In some embodiments, the vascular access system 10 may be configured both wirelessly and in a wired configuration with certain blood characteristics and/or parameters being measure or determined wirelessly and other blood characteristics and/or parameters being measured or determined with the wired configuration. In some embodiments, the wireless adapter 60 may be configured to transmit sensor data from the sensors 22 to the base unit 36 or possibly to the monitoring device 38. In some embodiments, the wireless adapter 60 may include batteries for powering the sensors 22. In some embodiments, the wireless adapter 60 may include one or more alerting mechanisms (e.g. LEDs, speakers, haptic units, etc.) to provide an alert. In some embodiments, the wireless adapter 60 may include a wireless board with one or more of a processor, communications, power, and sensor lead wires.

In some embodiments, the vascular access system 10 may include a protective tube 62 extending from the sensing probe hub 30. In some embodiments, the protective tube 62 may be constructed of polyimide or another suitable material. In some embodiments, the sensing probe 18 may be disposed within the protective tube 62, which may protect the sensing probe 18. In these and other embodiments, the sensing probe 18 may include multiple sensor lead wires. In some embodiments, an inner surface of the protective tube 62 may be spaced apart from the sensing probe 18 such that fluid, such as blood or another liquid, for example, may flow between the protective tube 62 and the sensing probe 18 and through the protective tube 62. In some embodiments, the protective tube 62 may include one or more fenestrations configured to pass air but not blood or liquid, which may facilitate movement of blood through the protective tube 62 between the protective tube 62 and the sensing probe 18.

In some embodiments, the sensing probe hub 30 may include a port 64. In some embodiments, the vascular access system 10 may include an extension tube 66 integrated with the port 64. In some embodiments, fluid may be configured to flow proximally through the protective tube 62 between the protective tube 62 and the sensing probe 18 and into the extension tube 66. Thus, in some embodiments, the vascular access system 10 may include a fluid pathway therethrough and may be used for blood sampling to measure blood characteristics not measured directly by the sensing probe 18. Additionally or alternatively, in some embodiments, the vascular access system 10 and/or the fluid pathway may be used for fluid or medication delivery. In some embodiments, a blood collection device may be coupled to a luer adapter 68 disposed at a proximal end of the extension tube 66. In some embodiments, a fluid chamber 70 may be formed within the sensing probe hub 30.

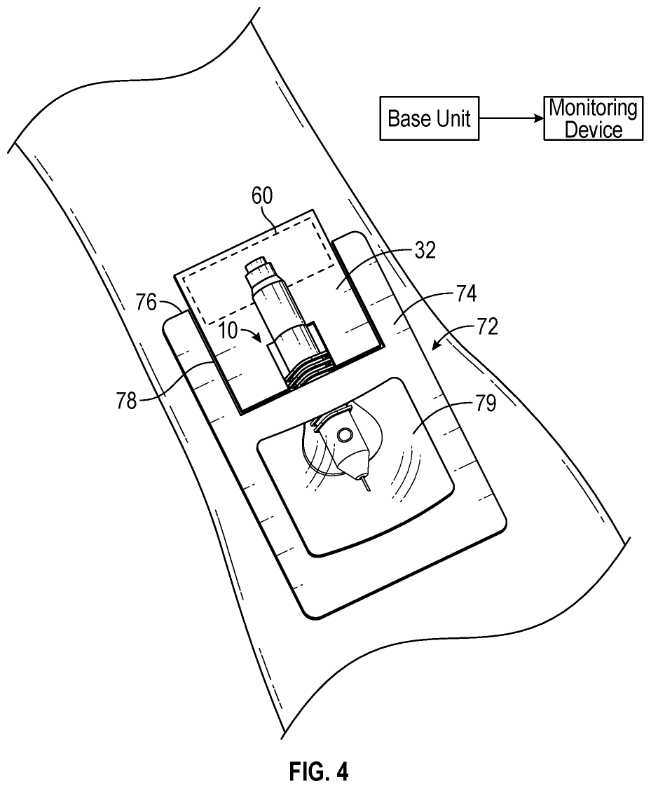

Referring now to FIG. 4, in some embodiments, the vascular access system 10 may be secured to skin of the patient with a securement dressing 72. In some embodiments, the securement dressing 72 may include a dressing material 74, which may provide an outside border around a perimeter of the securement dressing 72. In some embodiments, the dressing material 74 may be flexible and/or woven to conform to the skin of the patient.

In some embodiments, a bottom surface of the dressing material 74 may include an adhesive configured to adhere to the skin of the patient. In some embodiments, a proximal end 76 of the dressing material 74 may include a cutout 78, which may allow the stabilization platform 32 and/or the wireless adapter 60 to be visible to a user. Thus, in some embodiments, the alert mechanisms of the wireless adapter 60 may be visible to the user and/or the stabilization platform 32 may be accessible to the user. In some embodiments, the stabilization platform 32 may be positioned within the cutout 78. In some embodiments, the stabilization platform 32 may be contacting and/or proximate the dressing material 74. In some embodiments, an enclosed window 79 within the dressing material 74. In some embodiments, the enclosed window 79 may include a transparent material and may be configured to be placed over an insertion site in the skin.

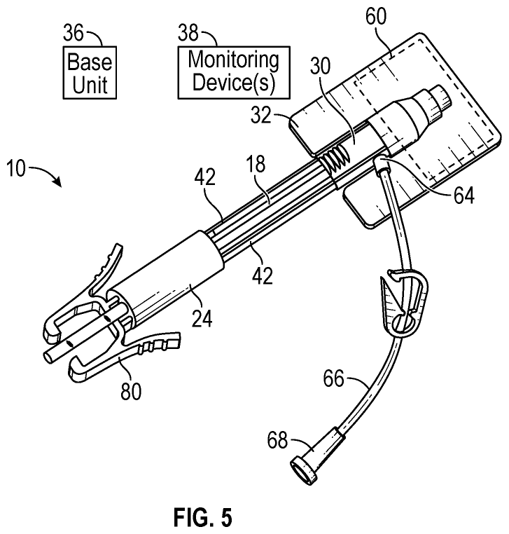

Referring now to FIG. 5, in some embodiments, the vascular access system 10 may not include the introducer needle 12 and instead may be configured to be coupled to a proximal end of a catheter assembly, such as, for example, an over-the-needle catheter assembly after removal of an introducer needle. In these embodiments, the distal end 28 of the housing 24 may include a connector 80 configured to couple with the catheter assembly. In some embodiments, the connector 80 may include a blunt plastic cannula and/or opposing lever arms for a clip connection. In some embodiments, the connector 80 may include a male luer. In some embodiments, after the vascular access system 10 is coupled to the catheter assembly, the sensing probe 18 may be advanced distally into a fluid path of the catheter assembly and/or through the catheter assembly into the vasculature.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A vascular access system, comprising:
an introducer needle, comprising a sharp distal tip, a proximal end opposite the sharp distal tip, and an introducer needle lumen extending through the sharp distal tip and the proximal end of the introducer needle; and
a sensing probe slidably disposed within the introducer needle lumen, wherein a distal end of the sensing probe is disposed within the introducer needle lumen in an initial configuration of the vascular access system, wherein the introducer needle is configured to retract in response to the distal end of the sensing probe being advanced beyond the sharp distal tip, wherein the distal end of the sensing probe comprises a sensor.

2. The vascular access system of claim 1, further comprising a housing comprising a longitudinal passage, wherein the introducer needle is slidably disposed within the longitudinal passage of the housing, wherein the introducer needle extends from a distal end of the housing in the initial configuration of the vascular access system.

3. The vascular access system of claim 2, further comprising a sensing probe hub, wherein the sensing probe extends distally from the sensing probe hub, wherein the introducer needle is fully retractable within the sensing probe hub and the housing when the sensing probe hub is fully advanced with respect to the housing.

4. The vascular access system of claim 3, wherein a proximal end of the sensing probe hub is coupled to or configured to couple to a cable configured to transmit data from the sensor.

5. The vascular access system of claim 3, wherein the vascular access system comprises a wireless adapter configured to transmit data from the sensor.

6. The vascular access system of claim 5, wherein the vascular access system further comprises a stabilization platform coupled to the sensing probe hub, wherein the wireless adapter coupled to the stabilization platform.

7. The vascular access system of claim 1, wherein the sensor is configured to measure a hemodynamic parameter.

8. The vascular access system of claim 1, further comprising a compressible sleeve surrounding the sensing probe and coupled to the sensing probe hub.

9. The vascular access system of claim 1, further comprising a sensing probe hub, wherein the sensing probe extends distally from the sensing probe hub, further comprising a protective tube extending from the sensing probe hub, wherein the sensing probe is disposed within the protective tube and spaced apart from the protective tube such that fluid may flow between the protective tube and the sensing probe.

10. The vascular access system of claim 9, wherein the sensing probe hub further comprises a port, further comprising an extension tube integrated with the port, wherein fluid is configured to flow proximally through the protective tube between the protective tube and the sensing probe and into the extension tube.

11. The vascular access system of claim 1, wherein the introducer needle is held in place in the initial configuration by a releasable latch, wherein the sensing probe is configured to release the releasable latch to allow the introducer needle to retract.

12. A method of venous or arterial sensing, the method comprising:
inserting an introducer needle of a vascular access system into a vein or an artery of a patient, wherein the vascular access system comprises:
the introducer needle, comprising a sharp distal tip, a proximal end opposite the sharp distal tip, and an introducer needle lumen extending through the sharp distal tip and the proximal end of the introducer needle; and
a sensing probe slidably disposed within the introducer needle lumen, wherein a distal end of the sensing probe is disposed within the introducer needle lumen in an initial configuration of the vascular access system, wherein the introducer needle is configured to retract in response to the distal end of the sensing probe being advanced beyond the sharp distal tip, wherein the distal end of the sensing probe comprises a sensor;
advancing the distal end of the sensing probe beyond the sharp distal tip such that the introducer needle is retracted, and the sensing probe remains in the vein or the artery; and
receiving sensor data from the sensor, wherein the sensor data comprises at least one blood characteristic.

13. The method of claim 12, wherein the at least one blood characteristic is selected from: blood pressure, temperature, pH, oxygen, blood gas, and oxygen saturation.

14. The method of claim 12, further comprising processing the data from the sensor to determine a parameter, wherein the parameter comprises an indication of an onset of a health complication or an indication of an onset of a health improvement.

15. The method of claim 14, wherein the parameter is determined using artificial intelligence.

16. The method of claim 12, wherein the vascular access system further comprises:
a sensing probe hub, wherein the sensing probe extends distally from the sensing probe hub;
a stabilization platform coupled to the sensing probe hub; and
a wireless adapter coupled to the stabilization platform and configured to transmit data from the sensor;
further comprising securing the vascular access system to skin of the patient with a securement dressing, wherein the securement dressing comprises:
a dressing material providing an outside border around the securement dressing, wherein a bottom surface of the dressing material comprises an adhesive configured to adhere to the skin of the patient, wherein a proximal end of the outside border comprises a cutout wherein the stabilization platform is positioned within the cutout; and
an enclosed window within the dressing material, wherein the enclosed window comprises a transparent material and is configured to be placed over an insertion site in the skin.

17. The method of claim 12, further comprising a sensing probe hub, wherein the sensing probe extends distally from the sensing probe hub, further comprising a protective tube extending from the sensing probe hub, wherein the sensing probe is disposed within the protective tube and spaced apart from the protective tube such that fluid may flow between the protective tube and the sensing probe.

18. The method of claim 17, wherein the sensing probe hub further comprises a port, further comprising an extension tube integrated with the port, wherein fluid is configured to flow proximally through the protective tube between the protective tube and the sensing probe and into the extension tube.

19. A vascular access system, comprising:
a housing, comprising a distal end and a proximal end, wherein the distal end comprises a connector configured to couple to a catheter assembly;
a sensing probe hub; and
a sensing probe extending distally from the sensing probe hub, wherein the sensing probe hub is configured to advance distal to the distal end of the housing in response to movement of the sensing probe hub towards the housing.

20. The vascular access system of claim 19, wherein the connector comprises a blunt plastic cannula or a male luer.
